(19) United States
(12) Patent Application Publication
(10) Pub. No.: US 2019/0259491 A1
(43) Pub. Date: Aug. 22, 2019

(54) INSTANCE LEVEL METADATA POPULATION OF A PACS DATABASE

(71) Applicant: INTERNATIONAL BUSINESS MACHINES CORPORATION, Armonk, NY (US)

(72) Inventors: Mark D. Bronkalla, Waukesha, WI (US); James Boritz, Toronto (CA)

(21) Appl. No.: 15/902,771

(22) Filed: Feb. 22, 2018

(51) Int. Cl. G16H 30/20; G16H 15/00; G06F 17/30; G06F 15/18

(52) U.S. Cl. CPC G16H 30/20; G16H 15/00; G06F 17/30271; G06F 15/18; G06F 17/30265

(57) ABSTRACT

Systems and methods for populating a target database with metadata for use in accessing medical image data. One system includes an electronic processor configured to, in response to a trigger event, query an archive database for instance level metadata of a plurality of image studies and store the instance level metadata of the plurality of image studies in the target database. The electronic processor is also configured to, based on an image study for a patient requested through an image viewer and at least one relevancy rule, determine a set of other image studies for the patient based on the instance level metadata stored in the target database and display information regarding the set of the other image studies for the patient within the image viewer without downloading image data for each image included in the set of the other image studies to the target database.

### TECHNICAL FIELD

Embodiments described herein relate to systems and methods for populating a PACS database. More specifically, embodiments described herein relate to populating a PACS database based on instance level metadata retrieved from at least one image archive.

### SUMMARY

A physician diagnosing a patient may order an image study using a particular imaging modality. Imaging modalities use various imaging techniques and hardware, including, for example, radiography, nuclear medicine, computed tomography (CT), mammography, molecular imaging, photoacoustic imaging, magnetic resonance imaging (MRI), echocardiography, magnetic particle imaging, elastography, tactile imaging, and ultrasound imaging.

Medical images collected as part of an imaging procedure and corresponding metadata are stored in an archive as a study. The image archive may be included in or communicate with a picture archiving and communication system (PACS), which controls access to images generated by one or more imaging modalities. A PACS may include a PACS server and a PACS database. The PACS database may store information regarding images stored in a remote archive, such as pointers, that may be used to retrieve an image from a particular remote archive. The PACS server may use the data stored in the PACS database to respond to requests for images from a viewer device submitted through an image viewer application (“image viewer”).

For example, a medical practitioner (for example, a reading physician) may use an image viewer to communicate with a PACS server and request an image study. The PACS server may respond to the request by providing location information for the requested image study, which the image viewer may use to directly access the images from the image archive. The medical practitioner may use the image viewer to review the retrieved images included in the requested study and, optionally, to generate a report containing findings for the image study.

When a medical practitioner retrieves and reviews medical images generated as part of a particular image study for a particular patient, the medical practitioner often also retrieves and reviews images generated as part of other image studies for the patient. For example, a reading physician may retrieve images generated as part of a previous image study to verify findings for a current image study or to better understand the images in the current image study. The previous image study may include images of the same or different anatomy as the current image study, images generated by the same or a different imaging modality as the current image study, or a combination thereof. For example, patients may have had multiple imaging studies performed at different times, which may or may not be related.

For example, FIG. 1 is a screenshot of an example user interface generated by an image viewer. As illustrated in FIG. 1, the user interface includes one or more thumbnails representing other image studies for a patient that may be related or relevant to the current image study being reviewed through the image viewer. Each thumbnail may be a representative image of an available image study or a series of images associated with an available image study, and the thumbnails may be organized or grouped in a logical fashion, such as chronologically. A medical practitioner may select (for example, drag and drop) a thumbnail to view the image study or series within the image viewer. In some embodiments, a textual listing of available image studies or series is displayed within the image viewer in addition to the thumbnails or as an alternative.

Various relevancy rules may be applied to determine the available image studies or series to list for a medical practitioner with respect a currently-viewed image study. These relevancy rules may consider the imaging modality used for the currently-viewed image study, the anatomy represented in the currently-viewed image study, a finding entered for the currently-viewed image study, or the like. Accordingly, to acquire sufficient data regarding other image studies available for a particular patient to apply the relevancy rules, other image studies may be prefetched, which involves downloading the entire contents (including image data) of a limited range of prior studies (for example, to the PACS database, the image viewer, or both). For example, when a medical practitioner is reading an image study for a particular patient, other images studies for the patient may be prefetched. Given the number of image studies for a particular patient, however, it may be difficult to identify what image studies to prefetch. Also, given the size of some image studies, only a limited number of studies may be prefetched. For example, typically only three to six image studies may be prefetched. Furthermore, prefetching may waste computing resources as an entire study may be downloaded that a reading physician never accesses. For example, conventional prefetching approaches rely on downloading entire studies and then parsing the studies for metadata, which may be used to generate the thumbnails or listings of studies as described above. Thus, this approach is costly in terms of bandwidth and computing requirements.

Alternatively or in addition, a PACS server may populate a PACS database with study level metadata (sometimes referred to providing a “half folder” mechanism). In particular, the PACS server retrieves patient demographics and study level metadata from the remote image archive and populates the PACS database based on the retrieved data. Accordingly, the PACS server may apply the relevancy rules as described above to this stored metadata to determine other image studies that may be relevant. However, even with this information, a medical practitioner typically needs to wait until an entire study is downloaded and processed (for example, parsing and processing of metadata for the images, series, or both included in the image study) before information regarding the image study can be provided to a medical practitioner (for example, in thumbnail form as described above). For example, an image viewer may apply various rules to image metadata to determine how to properly hang a study or an image and how a user can navigate through the images. Accordingly, populating a new PACS database using a “half folder” mechanism still results in delay and wasting of computing and bandwidth resources.

Thus, embodiments described herein populate a PACS database with instance level metadata (sometimes referred to herein as providing a “full folder” population), which can be used to display a useful depth of information to a medical practitioner regarding other image studies for a patient without requiring a full download of individual image studies or series and the computing and bandwidth resources associated with such a download. As described in more detail below, this mechanism retrieves (in addition to patient demographics and study level metadata as described above for the “half folder” mechanism) instance level information (metadata) for image studies of a particular patient from an existing image archive and populates the PACS database with the retrieved information. Accordingly, the PACS database, once populated, maintains the locations (references or pointers) to the instances (images, presentation states, key objects, reports, scanned documents, measurements, etc.), which provide an efficient image retrieval mechanism without the need to query multiple devices. Having the full set of instance level metadata also provides the information needed for efficient “hanging” or display of studies so that the images are arranged (and optionally downloaded) in an efficient and pleasing manner for the reading physician. Thus, by cataloguing not merely the study level metadata but also the instance level metadata, rules can be executed against the retrieved metadata to determine how to properly hang the study, to determine the order of retrieval of the images to the workstation for background loading, to allow a user to navigate the study at will (or at random), and the like without having to wait for the entire study (including one or hundreds of images) to be downloaded and metadata to be parsed after the download is complete. Furthermore, as compared to conventional prefetching as described above, the “full folder” population method described in the present application catalogues metadata for the entire set of studies for a patient (which may be dozens or hundreds of studies), which allows a user to organize and navigate through image studies quickly and efficiently. In particular, the metadata can be used to generate the thumbnails and listing of available image studies as described above, which allows a user to view sufficient detail regarding other image studies (including potentially all other image studies for a particular patient), without requiring a full download of any of the individual image studies.

For example, one embodiment provides a system for populating a target database with metadata for use in accessing medical image data. The system includes an electronic processor communicatively coupled to a memory. The memory stores instructions that, when executed by the electronic processor, cause the electronic processor to, in response to a trigger event, query an archive database via a communication interface for instance level metadata of a plurality of image studies based on at least one query rule and store the instance level metadata of the plurality of image studies received from the archive database in the target database. The memory also stores instructions that, when executed by the electronic processor, cause the electronic processor to, based on an image study for a patient requested through an image viewer and at least one relevancy rule, determine a set of other image studies for the patient based on the instance level metadata stored in the target database for the plurality of image studies and display information regarding the set of the other image studies for the patient within the image viewer based on the instance level metadata stored in the target database without downloading image data for each image included in the set of the other image studies to the target database.

Another embodiment provides a method of populating a target database with metadata for use in accessing medical image data. The method includes, in response to a trigger event, querying, with an electronic processor, an archive database via a communication interface for instance level metadata of a plurality of image studies based on at least one query rule and storing, with the electronic processor, the instance level metadata of the plurality of image studies received from the archive database in the target database. The method also includes, based on an image study for a patient requested through an image viewer and at least one relevancy rule, determining a set of other image studies for the patient based on the instance level metadata stored in the target database for the plurality of image studies and displaying information regarding the set of other image studies for the patient within the image viewer based on the instance level metadata stored in the target database without downloading image data for each image included in the set of other image studies to the target database.

Yet another embodiment provides non-transitory computer-readable medium storing instructions that, when executed by an electronic processor, perform a set of functions. The set of functions including, in response to a trigger event, querying an archive database via a communication interface for instance level metadata and series level metadata of a plurality of image studies based on at least one query rule and storing the instance level metadata and the series level metadata of the plurality of image studies received from the archive database in a target database. The set of functions also includes, based on an image study for a patient requested through an image viewer and at least one relevancy rule, determining a set of other image studies for the patient based on the instance level metadata and the series level metadata stored in the target database for the plurality of image studies and displaying information regarding the set of other image studies for the patient within the image viewer based on the instance level metadata and the series level metadata stored in the target database without downloading image data for each image included in the set of other image studies to the target database.

Other aspects of the invention will become apparent by consideration of the detailed description and accompanying drawings.

### BRIEF DESCRIPTION OF THE DRAWINGS

FIG. 1 is a screenshot illustrating a user interface displayed by an image viewer according to one embodiment.

FIG. 2 schematically illustrates a system for populating a PACS database according to one embodiment.

FIG. 3 is a flow chart illustrating an instance level population method for a PACS database performed by the system of FIG. 2.

### DETAILED DESCRIPTION

One or more embodiments are described and illustrated in the following description and accompanying drawings. These embodiments are not limited to the specific details provided herein and may be modified in various ways. Furthermore, other embodiments may exist that are not described herein. Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way, but may also be configured in ways that are not listed. Furthermore, some embodiments described herein may include one or more electronic processors configured to perform the described functionality by executing instructions stored in a non-transitory, computer-readable medium. Similarly, embodiments described herein may be implemented as non-transitory, computer-readable medium storing instructions executable by one or more electronic processors to perform the described functionality. As used in the present application, “non-transitory computer-readable medium” comprises all computer-readable media but does not consist of a transitory, propagating signal. Accordingly, a non-transitory computer-readable medium may include, for example, a hard disk, a CD-ROM, an optical storage device, a magnetic storage device, a ROM (Read Only Memory), a RAM (Random Access Memory), register memory, a processor cache, or any combination thereof.

In addition, the phraseology and terminology used herein is for the purpose of description and should not be regarded as limiting. For example, the use of “including,” “containing,” “comprising,” “having,” and variations thereof herein is meant to encompass the items listed thereafter and equivalents thereof as well as additional items. The terms “connected” and “coupled” are used broadly and encompass both direct and indirect connecting and coupling. Further, “connected” and “coupled” are not restricted to physical or mechanical connections or couplings and can include electrical connections or couplings, whether direct or indirect. In addition, electronic communications and notifications may be performed using wired connections, wireless connections, or a combination thereof and may be transmitted directly or through one or more intermediary devices over various types of networks, communication channels, and connections. Moreover, relational terms such as first and second, top and bottom, and the like may be used herein solely to distinguish one entity or action from another entity or action without necessarily requiring or implying any actual such relationship or order between such entities or actions.

As noted above, embodiments described herein provide an instance level or “full folder” population method for a PACS database. The method may retrieve instance level information (metadata) for image studies of a particular patient from an existing archive based on one or more triggering events. The stored instance level metadata provides information needed for efficient “hanging” or display of studies so that the images are arranged (and optionally downloaded) in an efficient and pleasing manner for a medical practitioner. In particular, by cataloguing not merely the study level metadata but also the instance level metadata, rules can be executed against the retrieved metadata to determine how to properly hang the study, to determine the order of retrieval of the images to the workstation for background loading, to allow a user to navigate the study at will (or randomly), and the like without having to wait for the entire study (including one or hundreds of images) to be downloaded and the metadata to be parsed after the download is complete. Thus, embodiments described herein provide novel mechanism for populating a PACS database to provide medical practitioners with a depth of information without consuming significant bandwidth and computing resources.

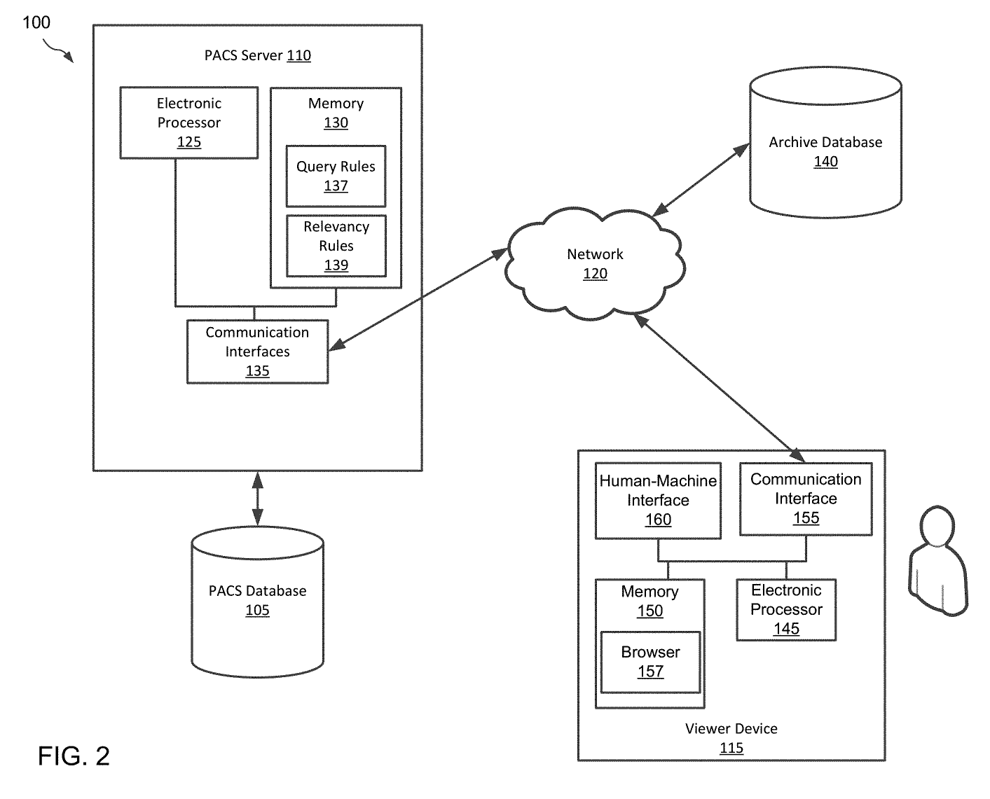

FIG. 2 schematically illustrates an example system 100 for populating a PACS database. As illustrated in FIG. 2, the system 100 includes a PACS database 105 (a target database), a PACS server 110, an archive database 140, and a viewer device 115. The PACS database 105, the PACS server 110, the viewer device 115, and the archive database 140 are communicatively coupled through a communication network 120. However, in other embodiments, the PACS database 105, the PACS server 110, the viewer device 115, and the archive database 140 communicate via one or more dedicated wire connections or other forms of wired or wireless electronic communication. It should be understood that the system 100 may include additional components than those illustrated in FIG. 2. For example, the system 100 may include multiple viewer devices 115, PACS servers 110, PACS databases 105, archive databases 140, or a combination thereof. The components of the system 100 may also communicate through one or more intermediary devices not illustrated in FIG. 2.

The PACS server 110 serves as a traffic controller for accessing medical images from the archive database 140 and providing the images to the viewer device 115 for display to a user. The PACS database 105 includes a memory, for example, a non-transitory, computer-readable medium, storing medical information such as medical studies, metadata, images, reports, or a combination thereof. In some embodiments, the PACS database 105 comprises a plurality of databases. Also, in some embodiments, the PACS database 105 is included in the PACS server 110. Although not illustrated in FIG. 2, the PACS database 105 may include a communication interface configured to communicate over the communication network 120 with the PACS server 110, and, optionally, other storage facilities, such as, for example, the archive database 140.

The archive database 140 is a medical image repository. For example, the archive database 140 may be a vendor neutral archive (VNA) or another suitable storage facility that stores medical image data generated by one or more different types of imaging modalities. The archive database 140 may comprise a single database system or a plurality of archive systems, which may be geographically dispersed. For example, one or more archive databases 140 may be used to populate the PACS database 105 whether the archive databases 140 are disparate systems or part of a business continuity or failover system. For example, in some embodiments, the archive database 140 includes geographically dispersed archives, wherein each archive contains a portion of the overall set of studies whether individually or in a partially replicated or redundant manner.

The archive database 140 stores one or more instances associated with one or more image studies. In general, an “instance” includes an object, such as an image, associated with an image study. For example, a still frame, a multi-frame sequence, or a movie generated as part of an image study may be referred to as an instance. Such image data may be formatted, stored, and transferred according to the digital imaging and communications in medicine (DICOM) format. Instances may also include non-image data, such as reports, scanned documents, or the like, that are encapsulated in a DICOM format or object. There are many types of DICOM objects, for example, images, reports, key image selections, key object selections, segmentation objects, registration objects, presentation states (typically comprising measurements or other annotations of images), and the like. Cross enterprise document sharing (XDS) objects or documents or other data formats or communication protocols may also be considered instances. XDS is an alternate storage mechanism that may be used in parallel to DICOM, such as for non-image documents and visible light images, such as skin photos or microscope images. Accordingly, the term “instance,” as used in the present application, includes all objects associated with a particular image study regardless of whether an object includes image data (pixel or voxel data).

While a one-to-one configuration of the PACS server 110 and the archive database 140 may be used as illustrated in FIG. 2, other configurations, such as a hub and spoke model, may be used. Furthermore, in some embodiments, the methods and systems described herein may be used to retrieve data from a new VNA, from an older PACS database, or another archive. Each study may include as few as a single instance (image) to many thousands of instances (images). Accordingly, in aggregate there may be many millions of studies and billions of instances in the system 100.

The PACS server 110 includes a plurality of electrical and electronic components that provide power, operational control, and protection of the components within the PACS server 110. For example, as illustrated in FIG. 2, the PACS server 110 includes an electronic processor 125, a memory 130, and communication interfaces 135. The electronic processor 125, the memory 130, and the communication interfaces 135 are communicatively coupled via one or more of a wireless connection, a dedicated wired connection, a communication bus, or the like. Although FIG. 2 only illustrates one PACS server 110, functionality performed by the PACS server 110 as described herein may be distributed among multiple servers, including servers providing a cloud service. In some embodiments, the PACS server 110 also performs other functionality in addition to the functionality described herein. Furthermore, the PACS server 110 may include additional components not shown in FIG. 2, such as one or more human-machine interfaces. In some embodiments, the PACS database 105 may be combined with the PACS server 110, the viewer device 115, or a combination thereof.

The electronic processor 125 may be a microprocessor, an application-specific integrated circuit (ASIC), or other suitable electronic device. The memory 130 includes non-transitory computer-readable medium, such as read-only memory (ROM), random access memory (RAM) (for example, dynamic RAM (DRAM), synchronous DRAM (SDRAM), and the like, electrically erasable programmable read-only memory (EEPROM), flash memory, a hard disk, a secure digital (SD) card, other suitable memory devices, or a combination thereof. The electronic processor 125 accesses and executes computer-readable instructions (“software”) stored in the memory 130. The software may include firmware, one or more applications, program data, filters, rules, one or more program modules, and other executable instructions. For example, the software may include instructions and associated data for performing a set of functions, including the methods described herein.

The communication interfaces 135 allow the PACS server 110 to communicate with devices external to the PACS server 110. For example, as illustrated in FIG. 2, the PACS server 110 may communicate with the PACS database 105, the viewer device 115, and other computing resources through the communication interfaces 135. The communication interfaces 135 may include a port for receiving a wired connection to an external device (for example, a universal serial bus (USB) cable and the like), a transceiver for establishing a wireless connection to an external device (for example, over one or more communication networks 120, such as the Internet, a local area network (LAN), a wide area network (WAN), and the like), or a combination thereof.

The viewer device 115 may be a desktop computer, a laptop computer, a workstation or terminal, a smartphone, a tablet computer, a smart television, a smart wearable, and the like. The viewer device 115 may include similar components as the PACS server 110. For example, as illustrated in FIG. 2, the viewer device 115 includes an electronic processor 145, memory 150, and communication interfaces 155, which may be communicatively coupled via a wireless connection, a dedicated wired connection, a communication bus, or the like. Although FIG. 2 only illustrates one viewer device 115, the system 100 may include multiple viewer devices. In particular, multiple viewer devices 115 may communicate with the PACS server 110 or the archive database 140 to access medical image study data and images from the PACS database 105 or the archive database 140. In some embodiments, the viewer device 115 performs other functionality in addition to the functionality described herein.

As illustrated in FIG. 2, the viewer device 115 may also include one or more human-machine interfaces 160. The human-machine interfaces 160 may include one or more input devices, such as a touch-screen, a mouse, a keyboard, a computer screen, a microphone, and the like, one or more output devices, such as a display device, a touchscreen, a speaker, a printer, or the like, or a combination thereof. It should be understood that the viewer device 115 may include additional components other than those illustrated in FIG. 2 in various configurations.

The viewer device 115 allows users to access images stored in the PACS database 105 or the archive database 140 through the PACS server 110 over a network connection, such as a webpage (image viewer) accessible over the Internet through a browser application 157. For example, the viewer device 115 may access a web server that authenticates users accessing the PACS server 110 over the Internet through the browser application 157 and, when authenticated, allows users to access and view images stored in the archive database 140. Alternatively or in addition, the viewer device 115 may store a dedicated image viewer application for communicating with the PACS database 105, the PACS server 110, the archive database 140, or a combination thereof for accessing images. A user may select an image study to access through a worklist displayed by the image viewer and may generate a report for an image study, such as a structured report that may be completed using text-to-speech technology, using the image viewer.

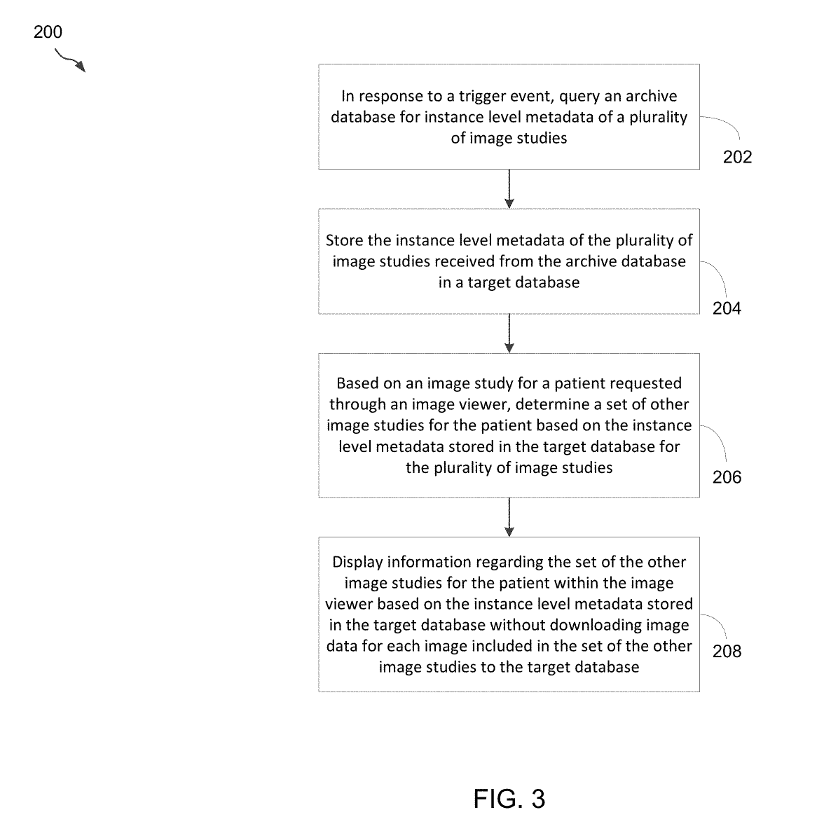

As noted above, the PACS database 105 may be initially populated with metadata using an instance level or “full folder on the fly” (FFOF) method. FIG. 3 illustrates a method 200 for performing such a FFOF method to populate the PACS database 105 with patient demographics, study level metadata, series level metadata, and instance level metadata for a set of image studies without downloading the corresponding image data (pixel or voxel data) for the set of image studies. In some embodiments, the method 200 is used to quickly populate the PACS database 105 with metadata from a new customer's existing archive (the archive database 140) when the new customer joins a PACS service. In particular, metadata regarding the customer's stored image data may be processed down to at least the instance level without fully processing the image data down to the image data (pixel or voxel) level. Accordingly, this processing may occur within a relatively short period of time, such as, for example, eight hours to a day depending on the amount of data. Thus, the method 200 allows the PACS service to quickly “go live” for a new customer.

The method 200 is described as being performed by the PACS server 110 (the electronic processor 125 executing instructions stored in non-transitory computer readable medium, such as the memory 130). However, it should be understood that the method 200 (or portions thereof) may be performed by other devices (the archive database 140, the viewer device 115) or distributed among multiple servers.

As illustrated in FIG. 3, the method 200 includes, in response to a trigger event, querying, with the PACS server 110, the archive database 140 for instance level metadata for a plurality of image studies (at block 202). The trigger event may include an order for a new study (an ORM message) for a patient, a patient admission (HL7 ADT) message for a patient, a receipt of an image study (new or old) for a patient, or the like. These trigger events may be detected based on the receipt of one or more messages or events from an electronic medical record (EMR) of a patient (for example, a scheduling event such as an upcoming office visit), a radiology information system (RIS), a hospital information system (HIS), or the like. For example, in some embodiments, the trigger event includes a DICOM or XDS transfer of an image study from an imaging modality. The trigger event may also be the opening of an image study for viewing within an image viewer. This trigger event may represent a final “catch up” query to catch any additions to the archive database 140 that have not been processed by the PACS server 110 yet. In addition, the trigger event may be a manual (or scheduled) trigger. For example, in some embodiments, the PACS database 105 is populated as a batch or bulk database import process as part of a data migration process. In some embodiments, multiple trigger events may occur simultaneously. In these situations, different priority rules may be applied to determine how to process data from the archive database 140. The priority rules can specify what types of triggers are associated with data processing that may be needed sooner than data processing associated with other types of data processing. For example, migration activity may be in progress while an image study is being viewed at a viewer device 115. In this situation, active viewing data communications may be prioritized and addressed ahead of any communications relating to the migration activity (which may otherwise continue to be addressed in a first-in-first-out (FIFO) manner), which reduces or entirely avoids a delay in the data retrieval performed for users. However, regardless of how the population is initiated, the population of the PACS database 105 as described herein provides similar benefits and improvements especially as patients have ever growing sets of archived studies (for example, dozens to many hundreds of studies).

In some embodiments, as illustrated in FIG. 2, the PACS server 110 executes query rules 137 to retrieve the instance level metadata. The query rules 137 may generate DICOM queries, WADO queries, or proprietary means to retrieve the instance level metadata. As described in more detail below, the query rules 137 may be configured or automatically learned using various machine learning techniques.

In some embodiments, the retrieved metadata also includes metadata regarding reports (DICOM SR objects, DICOM PDF objects, or DICOM CAD SR objects), XDS objects or documents, or other types of data. For example, for XDS objects or documents, the PACS server 110 may be configured to retrieve instance level metadata from one or a mix of a document registry and repository actors or devices. Given the size of such objects (non-image data objects), the PACS server 110 may retrieve both metadata for these objects as well as the objects themselves. For example, reports for any image studies represented by retrieved metadata may also be retrieved or pushed to the PACS server 110, such as from the archive database 140, an electronic medical record (EMR) of the patient, a radiologist information system (RIS), a report repository or the like. Such reports may be communicated using HL7 messaging (for example, medical document management (MDM) messages, observation result (ORU) messages, or the like). In some embodiments, reports that do not have existing image study entries in the archive database 140 may also be retrieved and used to populate the PACS database 105.

The instance level metadata includes pointers to image data stored in the archive database 140 (SOP instance unique identifier (UID)) as well as other information. This other information includes but is not limited to instance or slice number, photometric interpretation (monochromel, RGB, YUV, or the like), image resolution, image or slice position (three-dimensional vector information), phase, laterality (right or left), body part examined, contrast use, time delay from injection, cardiac cycle phasing, time delay from an excitation event, orientation such as axial, sagittal coronal, orientation via a position vector, MR acquisition sequence information, PET or NM attenuation correction type, laterality, view or orientation, angulation of the detector or x-ray geometry, ECG gating, respiratory gating, ultrasound acquisition type, presence of Key object selection, presentation state references, segmentation object references, registration object references DICOM SR object references, or the like. This information is typically used by an image viewer to organize and split the instance data into logical or physiological groupings presented via hanging protocols or default display layouts. As noted above, each image study may include as few as a single instance to many thousands of instances (images). Accordingly, the amount of metadata retrieved for a patient may include any amount of data.

Instance level metadata may optionally include series level metadata. For example, each image study includes one or more series, and each series includes one or more instances. A series may include a set of instances (images) generated as part of an image acquisition during the image study, such as images taken from a particular point of view or using a particular imaging procedure or technology. Similarly, a series may include a set of instances (images) generated from other images generated as part of an image study. For example, image data generated as part of an image study may be reformatted to change the view or other parameter of the images and these reformatted images may be stored as a separate series for the image study. Accordingly, when retrieving instance level metadata, series level metadata may be automatically retrieved as part of the instance level metadata. However, in other embodiments, series level metadata may be separately queried and retrieved. Series level metadata may include a series description, a series instance UID, contrast use information (multiple potential fields for type, injection or ingestion site, volume, and the like), modality (for example, CR, DX, MR, CT, and the like), acquisition sequence and protocol information, and the like.

Returning to FIG. 3, the method 200 also includes storing, with the PACS server 110, the instance level metadata of the plurality of image studies in the PACS database 105 (at block 204). Accordingly, once populated, the PACS database 105 maintains the locations or references (pointers) to all of the instances stored in one or more archive database 140 and, optionally, other systems and devices storing medical information, which provides efficient image retrieval without the need to query multiple devices and without having downloaded any image data (pixel or voxel data) to the PACS database 105. Having the full set of at least instance level metadata also provides information needed for efficient “hanging” or display of image studies so that the images are arranged in an efficient and pleasing manner for the reading physician. The manner in which a study (or individual images included in the image) is displayed or hung is a mix of the type of study and the images the study contains, the image acquisition series information, and, optionally, user preferences. Not hanging a study correctly may result in reduced user performance and, thus, inefficient use of medical resources. It should be understood that this use of the populated metadata is distinct from conventional DICOM Query and Retrieve (Q&R) mechanisms where when a study is retrieved where the metadata for the study has not yet been catalogued from a source PACS or archive, the user must typically wait for the entire study to be retrieved and the metadata to be parsed so that the study can be optimally organized or “hung” on a display device of a viewer device 115.

In other words, the metadata populated in the PACS database 105 allows instances in each prior or comparison image study for a patient to be characterized without the need to transfer the entire bulk of the images for all of the studies from the archive database 140. For example, as illustrated in FIG. 3, when an image study for a particular patient is requested for display within the image viewer, the PACS server 110 automatically determines a set of other image studies for the patient based on the instance level metadata (and optional series level metadata) stored in the PACS database 105 (at block 206) and displays information regarding the set of the other image studies for the patient within the image viewer based on the instance level metadata (and optional series level metadata) stored in the PACS database 105 without downloading image data for each image included in the set of other image studies to the PACS database 105 (at block 208).

In particular, the metadata may be used to group images (into series or subseries), arrange and cache images in an optimal order, and navigate through images in an ad hoc manner. For example, the metadata allows the sequencing of the retrieval of the images to be optimized to properly hang or display the images filling the image screen layout and thumbnail images initially and fill in the balance of the study behind the scenes. In addition, having the series and instance level metadata, a medical practitioner can rapidly navigate a study in an ad hoc manner (based on sequential order, orientation, or other factors) with instance retrievals being guided by actions of the medical practitioner and not merely based on a bulk study retrieval of all images which more likely than not is in a non-optimum sequence for the practitioner's current viewing.

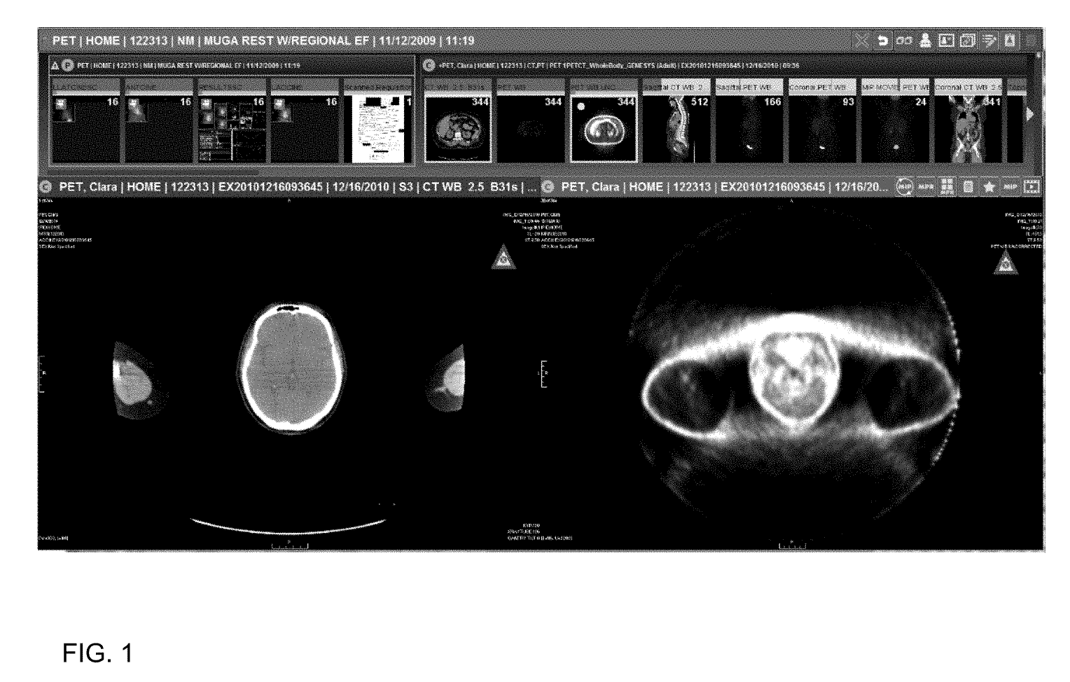

For example, based on the metadata, images can be grouped into series (including sub-series) and represented as thumbnails displayed within a pick list as illustrated in FIG. 1. In some embodiments, the PACS server 110 uses the metadata stored in the PACS database 105 to select a representative image for the thumbnail and uses the pointer of the selected image stored in the PACS database 105 to access image data from the archive database 140 and generate the thumbnail on the fly. Again, such thumbnails, lists, and picklists, and the like are provided without having done a prefetch of the entire image data for a particular image study.

In addition, when a user requests a particular instance (an image) based on the displayed information regarding the other studies, the pointer stored in the PACS database 105 allows the viewer device 115 to quickly access the requested instance. When the archive database 140 includes geographically dispersed archives storing duplicates of image data, the viewer device 115 may be configured to select the archive to retrieve image data from, such as from the closest or fastest archive. Similarly, in the event of a failure of a particular archive, the viewer device 115 may be configured to choose an alternate source for image data. It should be understood that, in some embodiments, the viewer device 115 receives image data from the archive database 140 through the PACS server 110. However, in other embodiments, the viewer device 115 receives image data from the archive database 140 (without interaction from the PACS server 110 other than providing the location (pointer) of the requested image).

As noted above, the PACS server 110 may implement one or more query rules 137 to determine what metadata to retrieve. In some embodiments, the query rules 137 may specify that metadata for all existing image studies for a patient should be retrieved from the archive database 140 or other medical information sources. As noted above, by retrieving instance level metadata but not the bulk image data for each instance, in many situations metadata can be retrieved and populated in the PACS database 105 for all image studies associated with a particular patient. However, in other embodiments, the query rules 137 may specify that only particular metadata is retrieved and used to populate the PACS database 105. For example, the rules may consider the trigger event, available image studies, a number of archive databases, or the like to determine the metadata to retrieve. In some embodiments, these rules are configurable to set particular rules for particular users (reading physician) or groups of users, patients, image study types, or the like.

Alternatively or in addition, the PACS server 110 may use a cognitive approach (using machine learning and various artificial intelligence techniques) to automatically learn what metadata to retrieve. For example, the PACS server 110 may record and learn what prior or comparison studies or instances users or groups of users request for particular types of image studies and use this training information to automatically determine query rules (a model) for retrieving metadata from the archive database 140. In particular, the PACS server 110 may track the types of studies accessed by a user as comparison studies, parts of anatomy imaged in studies assessed for comparison studies by a user, a role of a user performing a comparison study, findings included in reports for image studies, or the like. For example, the system 100 may use machine learning to build complex query rules that may be applicable to multi-site systems or cross-enterprise queries. Machine learning generally refers to the ability of a computer program to learn without being explicitly programmed. In some embodiments, a computer program is configured to construct a model (for example, one or more algorithms) based on example inputs. Supervised learning involves presenting a computer program with example inputs and their desired (for example, actual) outputs. The computer program is configured to learn a general rule (for example, a model) that maps the inputs to the outputs. The computer program may be configured to perform deep machine learning using various types of methods and mechanisms. For example, the computer program may perform deep machine learning using decision tree learning, association rule learning, artificial neural networks, inductive logic programming, support vector machines, clustering, Bayesian networks, reinforcement learning, representation learning, similarity and metric learning, sparse dictionary learning, and genetic algorithms. Using all of these approaches, a computer program may ingest, parse, and understand data and progressively refine models for data analytics.

Thus, embodiments described herein provide methods and systems for populating a PACS server with metadata, down to the instance level, quickly and efficiently, which allows for efficient organization and display of prior or comparison image studies without the need to prefetch the entire bulk image data for a study.

Various features and advantages are set forth in the following claims.

What is claimed is:

1. A system for populating a target database with metadata for use in accessing medical image data, the system comprising:
an electronic processor communicatively coupled to a memory, the memory storing instructions that when executed by the electronic processor, cause the electronic processor to:
in response to a trigger event, query an archive database via a communication interface for instance level metadata of a plurality of image studies based on at least one query rule;
store the instance level metadata of the plurality of image studies received from the archive database in the target database;
based on an image study for a patient requested through an image viewer and at least one relevancy rule, determine a set of other image studies for the patient based on the instance level metadata stored in the target database for the plurality of image studies; and
display information regarding the set of other image studies for the patient within the image viewer based on the instance level metadata stored in the target database without downloading image data for each image included in the set of other image studies to the target database.

2. The system of claim 1, wherein the trigger event includes at least one selected from a group consisting of an order message, an admission message, and a scheduling event.

3. The system of claim 1, wherein the trigger event includes a receipt of a new image study for the patient.

4. The system of claim 1, wherein the trigger event includes a manual trigger.

5. The system of claim 1, wherein the electronic processor is further configured to, in response to receiving a second trigger event, apply at least one priority rule to prioritize the trigger event and the second trigger event.

6. The system of claim 1, wherein the trigger event includes an opening of the image study of the patient within the image viewer.

7. The system of claim 1, wherein the at least one query rule queries the archive database for all image studies for the patient.

8. The system of claim 1, wherein the at least one query rule queries the archive database based on at least one preference.

9. The system of claim 1, wherein the at least one query rule is developed using machine learning and queries a plurality of archive databases including the archive database.

10. The system of claim 1, wherein the instance level metadata includes series level metadata.

11. The system of claim 1, wherein the electronic processor is further configured to query the archive database via the communication interface for series level metadata of the plurality of image studies in response to the trigger event and store the series level metadata in the target database, and wherein the electronic processor is configured to determine the set of other image studies for the patient based on the instance level metadata and the series level metadata stored in the target database for the plurality of image studies and is configured to display the information regarding the set of other image studies for the patient within the image viewer based on the instance level metadata and the series level metadata stored in the target database.

12. The system of claim 1, wherein the electronic processor is configured to display the information regarding the set of other image studies for the patient within the image viewer based on the instance level metadata stored in the target database by displaying instance level metadata for at least one image study included in the set of other image studies.

13. The system of claim 1, wherein the electronic processor is configured to display the information regarding the set of other image studies for the patient within the image viewer based on the instance level metadata stored in the target database by generating a thumbnail for at least one image study included in the set of other image studies based on the instance level metadata stored in the target database and image data stored in the archive database and displaying the thumbnail within the image viewer.

14. The system of claim 13, wherein the electronic processor is further configured to receive a selection of the thumbnail via the image viewer and return to the image viewer a pointer to at least one image stored in the archive database.

15. The system of claim 1, wherein the electronic processor is configured to display the information regarding the set of other image studies for the patient within the image viewer based on the instance level metadata stored in the target database by grouping images included in at least one image study included in the set of other image studies into a series of images and displaying a representation of the series of images within the image viewer for selection by a user.

16. A method of populating a target database with metadata for use in accessing medical image data, the method comprising:
in response to a trigger event, querying, with an electronic processor, an archive database via a communication interface for instance level metadata of a plurality of image studies based on at least one query rule;
storing, with the electronic processor, the instance level metadata of the plurality of image studies received from the archive database in the target database;
based on an image study for a patient requested through an image viewer and at least one relevancy rule, determining a set of other image studies for the patient based on the instance level metadata stored in the target database for the plurality of image studies; and
displaying information regarding the set of other image studies for the patient within the image viewer based on the instance level metadata stored in the target database without downloading image data for each image included in the set of other image studies to the target database.

17. The method of claim 16, wherein querying the archive database in response to the trigger event includes querying the archive database in response to receipt of at least one selected from a group consisting of an order message, an admission message, a new image study for the patient, a scheduling event, a manual trigger, and an opening of the image study of the patient within the image viewer.

18. The method of claim 16, further comprising querying the archive database via the communication interface for series level metadata of the plurality of image studies in response to the trigger event and storing the series level metadata in the target database, and wherein determining the set of other image studies for the patient includes determining the set of other image studies for the patient based on the instance level metadata and the series level metadata stored in the target database for the plurality of image studies and wherein displaying the information regarding the set of other image studies for the patient within the image viewer includes displaying the information regarding the set of other image studies for the patient based on the instance level metadata and the series level metadata stored in the target database.

19. Non-transitory computer-readable medium storing instructions that, when executed by an electronic processor, perform a set of functions, the set of functions comprising:
in response to a trigger event, querying an archive database via a communication interface for instance level metadata and series level metadata of a plurality of image studies based on at least one query rule;
storing the instance level metadata and the series level metadata of the plurality of image studies received from the archive database in a target database;
based on an image study for a patient requested through an image viewer and at least one relevancy rule, determining a set of other image studies for the patient based on the instance level metadata and the series level metadata stored in the target database for the plurality of image studies; and
displaying information regarding the set of other image studies for the patient within the image viewer based on the instance level metadata and the series level metadata stored in the target database without downloading image data for each image included in the set of other image studies to the target database.

20. The computer-readable medium of claim 19, wherein displaying the information regarding the set of other image studies for the patient within the image viewer based on the instance level metadata and the series level metadata stored in the target database includes grouping images included in at least one image study included in the set of other image studies into a series of images and displaying a representation of the series of images within the image viewer for selection by a user.
